(19) United States
(12) Patent Application Publication
(10) Pub. No.: US 2018/0071238 A1
(43) Pub. Date: Mar. 15, 2018

(54) Compositions and Methods for Traumatized Tissues Using Zinc Chelators

(71) Applicant: Ohio University, Athens, OH (US)

(72) Inventor: Yang V. Li, Athens, OH (US)

(73) Assignee: Ohio University, Athens, OH (US)

(21) Appl. No.: 15/561,632

(22) PCT Filed: Apr. 8, 2016
(86) PCT No.: PCT/US16/26587

§ 371 Date: Sep. 26, 2017

Related U.S. Application Data
(60) Provisional application No. 62/145,571, filed on Apr. 10, 2015.

(51) Int. Cl. A61K 31/198; A61K 31/7004; A61K 9/00; A61K 45/06

(52) U.S. Cl. CPC A61K 31/198; A61K 31/7004; A61K 2300/00; A61K 45/06; A61K 9/0085

(57) ABSTRACT

Neuroprotective compositions, methods of making the same, and methods of using the same, are disclosed.

### RELATED APPLICATIONS

This application claims priority to U.S. Provisional Application Ser. No. 62/145,571 filed under 35 U.S.C. § 111(b) on Apr. 10, 2015, the disclosure of which is incorporated herein by reference in its entirety.

### STATEMENT REGARDING FEDERALLY SPONSORED RESEARCH

This invention was made with no government support. The government has no rights in this invention.

### BACKGROUND OF THE INVENTION

Traumatic brain injury (TBI), or open TBI, is damage to the brain by external physical force which results in breaking the scalp/skull and damages specific parts of the brain. TBI or open TBI causes immediate brain injury, and, if it is not treated correctly, causes secondary brain injury in the neighboring neurons or brain regions. Therefore, TBI causes not only short-term brain injury, but also long-term health concerns. Many people live with TBI-related disabilities.

Currently, little is known about the cause of neuronal injury/death, other than direct mechanical damage. The target of treatment is typically limited to supportive procedures or cares. Importantly, there is no therapeutic treatment or intervention to prevent neuronal death (or brain damage) from the secondary brain injury after TBI or open TBI. Current treatments include simply continuous irrigation of the injured area or brain with physiological saline, surgical removal of debris (debridement) to reduce injury, or removal of pieces of the skull to relieve pressure inside the skull. After TBI, there is a general increase of intracranial (or cerebral) pressure due to accumulated cerebral spinal fluid, and, essentially, tissue swelling. Surgery is often needed to remove the swollen tissues. Methods that can limit brain damage and prevent the secondary brain injury would improve wound healing and the outcomes of the patients after TBI. Thus, there is a need in the art for new and improved compositions and methods for treating or preventing neuronal death from brain injuries.

### SUMMARY OF THE INVENTION

Provided herein is a neuroprotective composition for treating a wound or injury, the composition including saline and a zinc chelator, where the composition is at a pH ranging from about 2 to about 7. In certain embodiments, the pH ranges from about 4 to about 5. In certain embodiments, the zinc chelator comprises CaEDTA. In certain embodiments, the neuroprotective composition is isotonic. In certain embodiments, the neuroprotective composition is hypertonic. In certain embodiments, the neuroprotective composition further includes glucose. In particular embodiments, the glucose is present in an amount sufficient to make the neuroprotective composition hypertonic. In particular embodiments, the glucose is present at a concentration of about 20% by weight.

In certain embodiments, the zinc chelator is selected from the group consisting of: ethylenediaminetetra-acetic acid (EDTA); 1,3-diaminopropane-N,N,N′,N′-tetraacetic acid (DTPA); N,N,N′,N′-tetrakis(2-pyrdiylmethyl) ethylenediamine (TPEN); 1,10-phenanthroline; clioquinol; diethyldithiocarbamate (DEDTC), 2,3-dimercapto-1-propanesulfonic acid (DMPS); ethylenediamine-N,N′-diacetic-N,N′-di-B-propionic acid (EDPA); 1,2-dimethyl-3-hydroxy-4-pyridinone (DMHP); 1,2-diethyl-3-hydroxy-4-pyridinone (DEHP); ethylmaltol (EM), 4-(6-Methoxy-8-quinaldinyl-aminosulfonyl)benzoic acid potassium salt (TFLZn); dithiozone; N-(6-methoxy-8-quinolyl)-para-toluenesulfonamide (TSQ); carnosine; deferasirox; trans-1,2-cyclohexane-diamine-N,N,N′,N′-tetraacetic acid (CyDTA); dihydroxyethylglycine (DHEG); 1,3-diamino-2-hydroxypropane-N,N,N′,N′-tetraacetic (DTPA-OH); ethylenediamine-N,N′-diacetic acid (EDDA); ethylenediamine-N,N′-dipropionic acid (EDDP); ethylenediamine-N,N′-bis(methylphosphonic) acid (EDDPO); N-hydroxy-ethylenediamine-N,N′,N′-triacetic acid (EDTA-OH); ethylenediaminetetra(methylenephosphonic) acid (EDTPO); N,N′-bis(2-hydroxybenzyl)ethylenediamine-N,N′-diacetic acid (HBED); hexamethylene-1,6-diaminetetraacetic acid (HDTA); hydroxyethyliminodiacetic acid (HIDA); iminodiacetic acid (IDA); methyl-EDTA, nitrilotriacetic acid (NTA); nitrilotripropionic acid (NTP), nitrilotrimethylenphosphonic acid (NTPO); 7,19,30-trioxa-1,4,10,13,16,22,27,33-octaazabicyclo[11,11,11] pentatriacontane (O-Bistren); triethylenetetraaminehexaacetic acid (TTHA); ethyleneglycol bis(2-aminoethyl ether)-N,N,N′,N′-tetraacetic acid (EGTA); dimercaptosuccinic acid (DMSA); deferoxamine; dimercaprol; zinc citrate; combinations of bismuth and citrate; penicilamine; succimer; etidronate; ethylenediamine-di (O-hydroxyphenylacetic acid) (EDDHA); trans-1,2-cyclohexanediaminetetraacetic acid (CDTA); N-(2-hydroxyethyl) ethylenedinitrilotriacetic acid (HEDTA); N-(2-hydroxyethyl) iminodiacetic acid (HEIDA); 9-(O-carboxyphenyl)-2,7-dichloro-4,5,-bis[bis(2-pyridylmethyl)-aminomethyl]-6-hydroxy-3-xanthone; 9-(O-carboxyphenyl)-4,5-bis[bis(2-pyridylmethyl)-aminomethyl]-6-hydroxy-3-xanthanone; 9-(O-carboxyphenyl-2-chloro-5-[2-{bis(2-pyridylmethyl)aminomethyl}-N-methylaniline]-6-hydroxy-3-xanthanone; calprotectin; zinc fingers; lactoferrin; ovotransferrin; conalbumin; salts thereof; and combinations thereof.

In certain embodiments, the neuroprotective composition includes a buffer that maintains the pH of the neuroprotective composition in the range of from about 2 to about 7. In certain embodiments, the neuroprotective composition includes a buffer that maintains the pH of the neuroprotective composition in the range of from about 4 to about 5.

In certain embodiments, the neuroprotective composition further includes an additive selected from the group consisting of: contrast agents, secondary chelators, antibiotics, antiseptics, antifungals, growth factors, nucleic acids, proteins, chemotherapeutic agents, vitamins, bone resorption inhibitors, stem cells, dyes, opacifying agents, drug delivery vehicles, diagnostic agents, materials designed to release ions (other than zinc), and other small molecule or biological drugs.

Further provided is a method for treating an open wound or injury, the method comprising the steps of dissolving a zinc chelator in a saline solution, adjusting the pH of the saline solution to a pH of from about 2 to about 7 to produce an acidic composition, and administering the acidic composition to an open wound or injury to treat the open wound or injury. In certain embodiments, the open wound or injury is a traumatic brain injury. In certain embodiments, the method reduces swelling in or around the open would or injury.

Further provided is a method of treating a traumatic brain injury, the method comprising the steps of lowering the pH of injured brain tissue, chelating free zinc ions present in the injured brain tissue with one or more zinc chelators, and allowing the pH of the injured brain tissue to return to neutral or near neutral. In certain embodiments, the method further includes treating the injured brain tissue with a hypertonic solution to reduce swelling of the injured brain tissue.

Further provided is a method for treating an open wound or injury, the method comprising the steps of covering a wounded area of tissue with material soaked in a neuroprotective composition disclosed herein for a first period of time, and, optionally, washing or irrigating the wounded area with physiological saline for a second period of time. In certain embodiments, the wounded area of tissue includes brain tissue. In certain embodiments, the swelling of the open wound or injury is reduced.

Further provided is a method for treating an injury, the method comprising the steps of washing an injury with a neuroprotective composition disclosed herein for a first period of time, and washing the injury with normal saline for a second period of time, to treat the injury. In certain embodiments, the injury is a traumatic brain injury (TBI).

Further provided is a method of preventing an infection, the method comprising the steps of washing an injury or open wound of a subject with a neuroprotective composition disclosed herein for a sufficient period of time to treat the injury or open wound, and treating the subject with an antibiotic to prevent an infection in the injury or open wound.

Further provided is a kit for treating a wound or injury, the kit comprising a first container housing a saline solution, and a second container housing a zinc chelator. In certain embodiments, the kit further includes a third container housing an acid or buffer system.

### BRIEF DESCRIPTION OF THE DRAWING

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1: Brain injury model comparing the continuous irrigation or washing of an injured brain with neutral pH saline to the continuous irrigation or washing of an injured brain with a neuroprotective composition containing an acidic pH saline.

FIGS. 2A-2B: Photographs showing cross-sections of the brain after TBI (FIG. 2A) and after regular physiological saline (pH˜7.4) perfusion for 4 hours (FIG. 2B). The arrow in each image indicates the direct impact area by the TBI. The brain area above the white dashed line is the damaged brain. Damaged areas of the brain are markedly swollen.

FIG. 3: Photograph showing a cross-section of the brain following TBI and perfusion with hypertonic saline containing 20% glucose. The arrow indicates the direct impact area by the TBI. Brain area above the white dashed line is the damaged brain. The area included in the upper dashed line (at upright corner) is the area that is directly hit or impacted by the TBI. The brain damage between the two dashed lines is likely caused by the secondary impact of TI. Damaged brain areas are markedly less swollen compared to the brain section shown in FIGS. 2A-2B.

FIG. 4: Photograph showing a cross-section of the brain following TBI and continuous perfusion with acidic, hypertonic saline (20% glucose, pH˜4.3) containing CaEDTA for 2 hours. After 2 hours, the brain was then continuously perfused with CaEDTA and hypertonic saline for another 2 hours. This photograph shows that brain damage from the TBI was limited to the direct impact area with little secondary injury.

FIG. 5: Chart showing that the neuroprotective composition reduces brain edema (or swelling) caused by traumatic brain injury, compared to a physiological saline control and a hypertonic saline at neutral pH.

FIG. 6: Chart showing that the neuroprotective composition reduces the infarct volume or brain damage area caused by traumatic brain injury, compared to a physiological saline control and a hypertonic salien at neutral pH.

FIGS. 7A-7C: Photographs (FIG. 7A) and microscopic images (FIG. 7B) showing zinc dissolution in acidic solution and zinc precipitation or crystallization in neutral or basic solution. The red arrows indicate precipitation. FIG. 7C shows microscopic images of zinc precipitation in neutral pH which was dissolved when the pH was adjusted to an acidic pH.

FIG. 8: Images of single HeLa cells used to test cell damage caused by zinc precipitation or crystallization at the single cellular level. A large amount of debris, fragmentations, and broken cells are visible. A massive vacuole is visible forming inside the cell in the images at pH˜7/8, indicating dying cells.

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this disclosure pertains.

One supportive procedure in response to a TBI is to continuously irrigate or wash the injured brain with physiological saline in order to remove blood clots, foreign objects, and tissue debris (debridement). Hypertonic saline is sometimes used to improve intracranial pressure by reducing tissue swelling. Antibiotics may be included in the wash saline to prevent infection. However, washing with physiological saline does not prevent neuronal death or secondary brain damage.

Described herein is a neuroprotective composition useful for treating an open wound or other injury, such as a traumatic brain injury (TBI) or open TBI. The neuroprotective composition includes acidic (low pH) saline, which may or may not be hypertonic, with a zinc chelator or metal ion chelating agent. The neuroprotective composition has a pH ranging from about 2 to about 7. In certain embodiments, the neuroprotective composition can reduce neuronal death and brain damage, and can prevent secondary brain damage. Though the composition is referred to herein as a “neuroprotective” composition due to its advantageous effects in treating traumatic brain injury, the usefulness of the composition is by no means limited to brain injuries. To the contrary, the composition is useful in treating any open wound, due to the presence of free cytoplasmic Zn²⁺ ions following an injury.

Healthy cells have a very low level of free zinc. Zinc homeostasis is meticulously and tightly regulated such that, unlike other metal ions, free zinc is essentially absent in the cytosolic fluid, with an estimated concentration being below picomolar range (˜1 pM). However, zinc is involved in the cellular signaling of apoptosis and necrosis. For the brain in particular, the accumulation of cytoplasmic zinc (Zn²⁺) is a ubiquitous component of the cell death cascade in neurological disorders such as traumatic brain injury and ischemic brain damage. The accumulation of intracellular Zn²⁺ precedes other morphological and molecular changes following neuronal damage.

TBI, or other injury, causes the accumulation of free zinc (Zn²⁺) inside neurons or cells. Metal ion Zn²⁺ is normally bound within proteins, leaving little free Zn²⁺. Damage to neurons or cells causes an increase in the free Zn²⁺ ions that results from the dissociation of Zn²⁺ from its binding proteins. TBI, as a particular example, disrupts brain function and metabolism, which in turn causes the dissociation of Zn²⁺ from its binding proteins. Therefore, there is an accumulation of free zinc inside neurons/cells in the TBI impacted brain area. These free zinc ions are soluble due to the acidic environment (low pH) in the TBI impacted brain area, or in other tissue damaged from a blunt force impact. When the acidic or low pH is corrected to normal neutral pH or higher, the accumulated free Zn²⁺ ions become insoluble Zn²⁺ particles or crystals. For example, at a pH of around 7, the zinc crystallizes and becomes insoluble. Without wishing to be bound by theory, it is believed that such zinc precipitation can cause neuron or cell death.

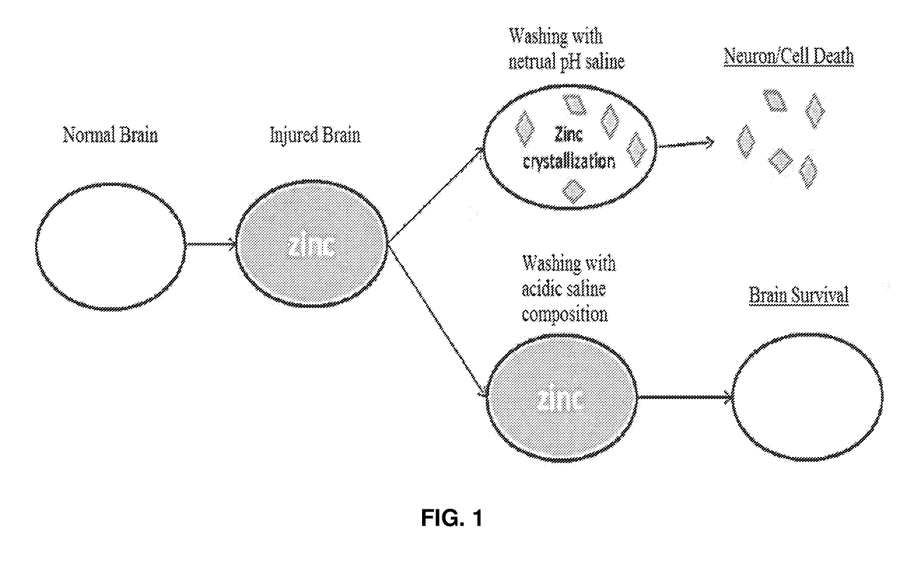

Continuously irrigating or washing damaged cells or neurons with physiological saline at neutral pH is a routine clinical practice. However, it can cause Zn²⁺ crystallization (Zn²⁺ precipitation), and consequently neuron/cell death. Thus, in accordance with the present disclosure, the injured brain or other area is irrigated or covered with saline at an acidic pH that keeps Zn²⁺ in a soluble state. This process is illustrated in FIG. 1. The acidic saline further includes one or more Zn²⁺ chelators which remove the free Zn²⁺ accumulated inside cells. The acidic saline and zinc chelator composition, referred to herein as a neuroprotective composition, can be applied continuously as a washing or irrigation solution, or can be used to cover the injured area. The acidic saline can keep the zinc in free form, while the chelators can remove the free Zn²⁺. In the brain, this prevents neuron/cell death and reduces brain damage. Elsewhere in the body, this prevent cell death and is useful in treating open wounds. Thus, the compositions and methods described herein can be applied to treat any blunt force tramatic wounds in any part of the body.

The neuroprotective composition includes one or more zinc chelators to chelate the free zinc (Zn²⁺) ions. Suitable zinc chelators include, but are not limited to: ethylenediaminetetra-acetic acid (EDTA); 1,3-diaminopropane-N,N,N′,N′-tetraacetic acid (DTPA); N,N,N′,N′-tetrakis(2-pyrdiylmethyl) ethylenediamine (TPEN); 1,10-phenanthroline; clioquinol; diethyldithiocarbamate (DEDTC), 2,3-dimercapto-1-propanesulfonic acid (DMPS); ethylenediamine-N,N′-diacetic-N,N′-di-B-propionic acid (EDPA); 1,2-dimethyl-3-hydroxy-4-pyridinone (DMHP); 1,2-diethyl-3-hydroxy-4-pyridinone (DEHP); ethylmaltol (EM), 4-(6-methoxy-8-quinaldinyl-aminosulfonyl)benzoic acid potassium salt (TFLZn); dithiozone; N-(6-methoxy-8-quinolyl)-para-toluenesulfonamide (TSQ); carnosine; deferasirox; trans-1,2-cyclohexane-diamine-N,N,N′,N′-tetraacetic acid (CyDTA); dihydroxyethylglycine (DHEG); 1,3-diamino-2-hydroxypropane-N,N,N′,N′-tetraacetic (DTPA-OH); ethylenediamine-N,N′-diacetic acid (EDDA); ethylenediamine-N,N′-dipropionic acid (EDDP); ethylenediamine-N,N′-bis(methylphosphonic) acid (EDDPO); N-hydroxy-ethylenediamine-N,N′,N′-triacetic acid (EDTA-OH); ethylenediaminetetra(methylenephosphonic) acid (EDTPO); N,N′-bis(2-hydroxybenzyl)ethylenediamine-N,N′-diacetic acid (HBED); hexamethylene-1,6-diaminetetraacetic acid (HDTA); hydroxyethyliminodiacetic acid (HIDA); iminodiacetic acid (IDA); methyl-EDTA, nitrilotriacetic acid (NTA); nitrilotripropionic acid (NTP), nitrilotrimethylenphosphonic acid (NTPO); 7,19,30-trioxa-1,4,10,13,16,22,27,33-octaazabicyclo[11,11,11] pentatriacontane (O-Bistren); triethylenetetraaminehexaacetic acid (TTHA); ethyleneglycol bis(2-aminoethyl ether)-N,N,N′,N′-tetraacetic acid (EGTA); dimercaptosuccinic acid (DMSA); deferoxamine; dimercaprol; zinc citrate; combinations of bismuth and citrate; penicilamine; succimer; etidronate; ethylenediamine-di (O-hydroxyphenylacetic acid) (EDDHA); trans-1,2-cyclohexanediaminetetraacetic acid (CDTA); N-(2-hydroxyethyl) ethylenedinitrilotriacetic acid (HEDTA); N-(2-hydroxyethyl) iminodiacetic acid (HEIDA); 9-(O-carboxyphenyl)-2,7-dichloro-4,5,-bis[bis(2-pyridylmethyl)-aminomethyl]-6-hydroxy-3-xanthone; 9-(O-carboxyphenyl)-4,5-bis[bis(2-pyridylmethyl)-aminomethyl]-6-hydroxy-3-xanthanone; 9-(O-carboxyphenyl-2-chloro-5-[2-{bis(2-pyridylmethyl)aminomethyl}-N-methylaniline]-6-hydroxy-3-xanthanone; calprotectin; zinc fingers; lactoferrin; ovotransferrin; conalbumin; salts thereof; and combinations thereof. In other embodiments, the zinc chelator is a wild-type apoenzyme or genetically engineered mutant thereof. Examples of the apoenzyme include, but are not limited to, apo-carbonic anhydrase, a S166C mutant, a H36C mutant, or a E117A mutant.

In particular embodiments, the zinc chelator is calcium disodium EDTA (CaEDTA). CaEDTA is particularly useful as the zinc chelator for several reasons. For instance, CaEDTA is more specific for zinc than other ions, such as calcium. Also, CaEDTA is an extracellular, membrane-impermeant chelator, meaning it generally does not cross the cellular membrane and remain inside cells.

The zinc chelator can be present in the composition at a concentration ranging from about 1 μM to about 100 mM. The zinc chelator is typically present in the composition at a concentration ranging from about 1 mM to about 20 mM. For example, the concentration of the zinc chelator can be about 5 mM. In other embodiments, the concentration of the zinc chelator in the neuroprotective composition ranges from about 10 μM to about 1000 μM. The concentration of the zinc chelator can be selected based on the desired use. For instance, the optimal concentration of zinc chelator present in a neuroprotective composition to be used on damaged brain tissue may be different from the optimal concentration of zinc chelator present in a neoroprotective composition to be used in an open wound not involving brain tissue.

If the pH of the neuroprotective composition is too low, it may cause denaturing of proteins in the brain or other anatomical location. Therefore, it is important that the pH of the neuroprotective composition range from about 2 to about 7, or, in some embodiments, from about 4 to about 5. Though the pH of the composition ranges from about 2 to about 7, the pH of the anatomical environment being treated with the composition is generally higher. When the composition is applied to the brain, for instance, the composition perfuses into the superficial layer of damaged tissue, where the desired pH range is from about 6 to about 7. Because the brain normally keeps producing spinal fluids, this pH of the superficial layer can be maintained by perfusing with a composition having a pH ranging from about 4 to about 5. In use, the neuroprotective composition interacts with the spinal fluids, and eventually these interactions neutralize the pH of the brain.

The pH of the neuroprotective composition can be adjusted through any of several methods. In some embodiments, the pH is adjusted by adding a sufficient amount of a strong acid until the pH reaches the desired level. Suitable strong acids include, but are not limited to: HCl, HI, HBr, HNO₃, H₂SO₄, and HClO₄. In other embodiments, the pH is adjusted by adding a sufficient amount of a weak acid until the pH reaches the desired level. Suitable weak acids include, but are not limited to: H₂CO₃, acetic acid (CH₃COOH), and oxalic acid (H₂C₂O₂).

In other embodiments, the pH of the neuroprotective composition is controlled by the inclusion of a suitable buffer system. A buffer system is a weak conjugate acid-base pair (i.e., either a weak acid and its conjugate base, or a weak base and its conjugate acid) that can resist a pH change upon the addition of an acidic or basic component, and is able to neutralize small amounts of added acid or base. Buffer systems are thus particularly useful for neuroprotective compositions which further include one or more additives that would otherwise materially affect the pH of the composition. Buffer systems work best when the pKₐ of the weak acid in the buffer system is close to the desired pH range. Suitable buffer systems for the neuroprotective composition include, but are not limited to: phosphoric acid/sodium phosphate monobasic; citric acid/sodium citrate; acetic acid/sodium acetate; a mixture of calcium carbonate and magnesium carbonate; and aqueous potassium hydrogen phthalate (KHP). However, many buffer systems can have biological effects. For example, at a high enough concentration, phosphate buffers may inhibit the activity of certain metabolic enzymes such as carboxylate, fumarase, and phosphoglucomutase. Therefore, the concentration of the buffer system used in the neuroprotective composition should be kept as low as possible to maintain the desired pH level.

In certain embodiments, the neuroprotective composition is hypertonic. By hypertonic, it is meant that the composition has a higher osmotic pressure than normal body cells, such that application of the composition to body cells would tend to draw water out of the cells by osmosis. The higher osmotic pressure than normal body cells can result from the composition having a higher salt concentration than normal body cells. However, it is understood that zinc can be removed from the injury site, and the injury can thereby by treated, without the neuroprotective composition being hypertonic.

Utilizing a hypertonic neuroprotective composition is beneficial for reducing swelling around a wound or injury. The desired level of osmality to create a hypertonic neuroprotective composition can be reached by adding any of various solutes to the composition. In some embodiments, glucose is included in the composition at a concentration of about 20% by weight. This results in the composition having a sufficient osmality to be hypertonic. Furthermore, the use of glucose to create a hypertonic composition is not harmful to the brain, as the normal brain requires, and can metabolize, glucose. Other concentrations of glucose can be used, so long as the resulting neuroprotective composition has an osmolality that makes it hypertonic.

In other embodiments, the neuroprotective composition is made hypertonic by an excess amount of sodium chloride or other salt. For instance, a saline solution containing from about 2% to about 3% sodium chloride w/v water is a hypertonic solution. In certain embodiments, the hypertonic solution includes saline containing from about 2.4% to about 2.6% w/v sodium chloride in water.

The neuroprotective composition may further include one or more pharmaceutically acceptable additives, based on the intended use of the composition. Suitable additives include, but are not limited to, contrast agents, such as Gadolinium contrast medium; secondary chelators, specific for ionic species (such as iron) other than zinc; antibiotics; antiseptics; antifungals; growth factors; nucleic acids; proteins; chemotherapeutic agents; vitamins; bone resorption inhibitors; stem cells; dyes; opacifying agents; drug delivery vehicles (whether loaded with drugs or not), such as liposomal formulations; diagnostic agents; materials designed to release ions, such as silver ions in the form of a colloidal silver; or other small molecule or biological drugs. The phrase “pharmaceutically acceptable” refers to molecular entities and compositions that produce no adverse, allergic, or other untoward reaction when administered to an animal, such as, for example, a human. The preparation of a pharmaceutically acceptable neuroprotective composition that contains at least one additive or additional ingredient will be known to those of skill in the art in light of the present disclosure, as exemplified by Remington's Pharmaceutical Sciences, 2003, incorporated herein by reference. Moreover, for animal (e.g., human) administration, it will be understood that preparations should meet sterility, pyrogenicity, and general safety and purity standards as required by the FDA Office of Biological Standards.

In some embodiments, the neuroprotective composition includes one or more antibiotics to prevent or treat an infection. Suitable antibiotics include, but are not limited to, penicillins, tetracyclines, glycylcyclines, macrolides, sulfonamides, cephalosporins, monobactams, carbapenems, aminoglycosides, quinolones, oxazolidinones, and combinations thereof. In certain non-limiting examples, the neuroprotective composition includes at least one antibiotic selected from the group consisting of clindamycin, tigecycline, vancomycin, ciprofloxacin, ofloxacin, sulfamethoxazole, trimethoprim/sulfamethoxazole, amoxicillin, penicillin V, penicillin G, procaine penicillin, benzathine penicillin, carbencillin, mezlocillin, ampicillin, piperacillin, bacampicillin, tiearcillin, ticarcillin, piperacillin/tazobactam, aztreonam, cefotetan, loracarbef, mefoxin, merrem, levofloxacin, lomefioxacin, primaxim, cycloserine, kanamycin, dicloxacillin, demeclocycline, minocycline, doxycycline, oxytetracycline, tobramycin, gentamicin, neomycin, amikacin, craramyein, nebcin, erythromycin/sulfisoxazole, netromycin, streptomycin, tobramycin, cefotaxime, cefuroxime, cefazoline, ceffibuten, ceffizoxime, cefaclor, cefopoerazone, cefprozil, cefadroxil monohydrate, ceftazidime, trimethoprim/sulfamethoxazole, cephalexin, cefazolin, cefamandole nafate, cefepime, cefonicid, sulfadiazine, norfloxacin, enoxacin, cefdinir, seromycin, ceftriaxone, cefixime, ceftazidime, clarithromycin, dirithromycin, methenamine, ethionamide, trovafioxacin, sparfloxacin, interfon-α, indinavir, ganciclovir, foscamet, lamivudine, famciclovir, rimantadine, zalcitabine, interferon-β, indinavir, ganciclovir, saquinavir, ritonavir, ribavirin, erythromycin, troleandomycin, azithromycin, eliiidamycin, colistin, amphotericin B, flucytosine, fluconazole, griseofulvin, grepafloxacin, ultramicrosize griseofulvin, terbinafine, ketoconazole, clotrimazole, dapsone, delavirdine, ziduvudine, amantadine, palivizumab, valacyclovir, didanosine, nelfinavir, nevirapine, ribavirin, cidofovir, pyrimethamine, metronidazole, furazolidone, atovaquone, stavudine, lamiduvine, acyclovir, mionazole, nystatin, itraconazole, chloroquine, pyrimethamine, mefloquine, hydroxychloroquine, capreomycin, permethrin, crotamiton, lindane, fluoro-uracil, ethambutol, rifabutin, isoniazid, aminosalicyclic acid, rifapentine, pyrazinamide, coenzoyl peroxide, chlorhexidine gluconate, sodium oxychlorosene, benzoyl peroxide, rifampin, rifampin/isoniazid, rifampin/isoniazid/pyrazinamide, nitrofurantoin, linezolid, nitrofurantoin, fosfomycin, nalidixic acid, atropine, oxytetracycline/sulfamethizole/phenazopyridine, mupirocin, chloramphenical, neomycin/polymyxin, tfimetorpim/polythyxin, tobramycin/dexamethasone, vidatabine, ciprofloxacin, ofioxacin, sulfacetamide, mupirocin, povidoneodine, gentamicin, nystatin, chloramphenicol, bacitracin, sulconazole, terbinafine, tetrachlorosalicylanilide, metronidazole, metromdazole, ciclopiroxolamine, clotrimazole, clotrimazole/betamethasone, butenafine, clotrimazole, nattifine, oxiconazole, selenium, econazole, penciclovir, or a pharmaceutically acceptable salt thereof.

Because the neuroprotective composition can be administered to skin, such as abraded skin, the neuroprotective composition may contain a penetration enhancer to facilitate adsorption of the active ingredients through the skin. Suitable penetration enhancers include, but are not limited to, glycerin, alcohols, alkyl methyl sulfoxides, pyrrolidones, and luarocapram. Possible bases for compositions for topical application include polyethylene glycol, lanolin, cold cream, and petrolatum as well as any other suitable absorption, emulsion, or water-soluble ointment base. Topical preparations of the neuroprotective composition may also include emulsifiers, gelling agents, and antimicrobial preservatives as necessary to preserve the neuroprotective composition and provide for a homogenous mixture. The neuroprotective composition should be stable under the conditions of manufacture and storage and may optionally be preserved against the contaminating action of microorganisms, such as bacteria and fungi. The prevention of the action of microorganisms can be brought about by various antibacterial and antifungal agents, for example, parabens, chlorobutanol, phenol, sorbic acid, thimerosal, and the like.

In certain embodiments, the neuroprotective composition is suitable for delivery by eye drops, where, without wishing to be bound by theory, it is believed zinc chelation may be beneficial. When the neuroprotective composition is formulated for delivery by eye drops, the composition may preferably be isotonic instead of hypertonic so as to prevent pain or discomfort. Furthermore, the pH of the eye is normally neutral (7.0 to 7.3); therefore, when the neuroprotective composition is formulated for delivery by eye drops, the pH of the neuroprotective composition is preferably in the upper end of the range of from about 3 to about 7, although still acidic, so as to prevent damage to the eye.

Further provided are methods of treating an open wound or other injuries with the neuroprotective composition described herein. Without wishing to be bound by theory, it is believed that all damaged skin and muscle cells release free zinc ions that can be removed with a neuroprotective composition containing one or more zinc chelators. The term “wound” is used to refer to an injury to the body, which may result from a disease or a disease-associated condition, burns or frost, infections, radiation, chemicals, violence, accident, or surgery that typically involves laceration or breaking of a membrane, including but not limited to the skin, and may involve damage to underlying surrounding tissues. The term “wound” includes acute wounds and chronic wounds, where chronic wounds include but are not limited to diabetic foot ulcers, venous and arterial leg ulcers, pressure ulcers, and wounds caused by ischemia or radiation. The term “wound” also includes both internal and external wounds, and includes open and closed wounds unless otherwise specified. The neuroprotective composition may be used for particularly advantage for the treatment of wounds, burns, infections, peritonitis, sepsis, for reducing wound hemorrhage, or for reducing scar formation. Methods for treating an open wound with the neuroprotective composition may optionally further include treating the wound with a suitable antiseptic, antibiotic, antimicrobial, or the like.

The neuroprotective composition can be continuously perfused into an open wound or open skull. The neuroprotective composition can be applied directly to, and used to treat, injured brain tissue, muscle, or skin. In some embodiments, a material, such as a bandage or cloth, is soaked in the neuroprotective composition and then placed directly on the injured tissue or wound for a desired period of time.

In various embodiments, following perfusion with the neuroprotective composition for a desired amount of time, the neuroprotective composition is replaced with normal saline for a desired amount of time. Without wishing to be bound by theory, it is believed that perfusing with normal saline following perfusion with the neuroprotective composition helps prevent possible injury from prolonged exposure to an acidic pH. In one non-limiting example, injured brain tissue or an open wound is continuously perfused with the neuroprotective composition for a first period of time ranging from about 1 minute to about 6 hours, such as about 2 hours, and then continuously perfused with normal saline for a second period of time ranging from about 1 minute to about 6 hours, such as about 2 hours. In other embodiments, the tissue or wound is continuously perfused with only the neuroprotective composition, for a time period ranging from about 1 minute to about 6 hours. The optimal duration of perfusion with the neuroprotective composition will vary according to factors such as the extent of the injury, the age and size of the patient, and the amount of time which has passed between the time the injury was sustained and the time the perfusion begins. Without wishing to be bound by theory, it is believed that the methods of treating a wound or injury with the neuroprotective composition are more advantageously employed as soon as possible following the injury, when the presence of free Zn²⁺ ions is higher. However, the neuroprotective composition may still be applied to a wound or brain tissue several hours, days, or even weeks following an injury.

In some situations, the neuroprotective composition may be used in the treatment of a wound where the treatment also requires the use of a bone cement composition to repair bone damaged at the site of the wound. In such scenarios, because many bone cements are set into a hardened mass upon an acid-base reaction (where the acid is generally provided as a setting solution), the acidic nature of the neuroprotective composition can cause the neuroprotective composition to act as a setting solution for any acid-base bone cement. Such treatment involves placing the base component of the bone cement in or on the damaged bone, and perfusing the area with the neuroprotective composition to both (i) cause the bone cement to set into a hardened mass, and (ii) remove free zinc ions from the damaged tissue in the wound surrounding the damaged bone.

Though the compositions and methods described herein can effectively remove free Zn²⁺ ions from damaged or injured tissue, zinc is nonetheless an integral component of thousands of different proteins, none of which are functional if the zinc is removed. Zinc metalloproteins include transcription factors containing zinc fingers, structural proteins, and enzymes from all six enzyme classes. Zinc metalloproteins are present in virtually every cellular organelle of every cell type in the brain and body. Zinc is also important for DNA stabilization and gene expression. Thus, zinc deficiency can be harmful, or even fatal in some situations. Without wishing to be bound by theory, it is believed that chelating zinc to too low a level can induce seizures or facilitate apoptosis. Therefore, the compositions and methods herein should be practiced with an understanding of, and respect for, the importance of zinc in the overall body. Accordingly, certain embodiments of the methods herein involve either monitoring free zinc levels in a subject undergoing the treatment, and/or supplementing zinc levels during or following the treatment.

As one non-limiting example, free zinc levels can be monitored through the use of zintrodes. Zintrodes are chemical sensors/probes based on optical fibers which can be used to transmit excitation light from an illumination source to an analyte captured within an area of the probe, and to collect the return signal, either emission or scattering, onto a detector. The fiber optic probe is small and enables in vitro or in vivo real-time monitoring of biological fluids and in vivo implantation. Typically, the zintrode body is made of an inert, biocompatible material, such as a fluorocarbon, with a port or an area in the wall that is fitted with a material permeable to the analyte (Zn²⁺), which reacts with a reagent contained within the body of the sensor.

As another non-limiting example, zinc levels can be supplemented through the administration of any suitable zinc supplement. Zinc supplements include, but are not limited to, one or more forms of inorganic or organic zinc, or combinations thereof, for oral or parenteral administration, including solid, gel, liposomal, and liquid forms. Non-limiting examples include zinc chloride (ZnCl₂); tetrabasic zinc chloride (Zn₅Cl₂(OH)₈); zinc oxide (ZnO); zinc sulfate (ZnSO₄); zinc proteinates; zinc chelates; zinc amino acid complexes, such as zinc histidine, zinc methionine, or zinc lysine complexes; zinc acetate; zinc ascorbate; zinc aspartate; zinc butyrate; zinc carbonate; zinc citrate; zinc gluconate; zinc glycinate; zinc histidinate; zinc lactate; zinc maleate; zinc picolinate; zinc propanoate; zinc stearate; and zinc succinate. In certain embodiments, the zinc supplements are administered to deliver from about 10 mg to about 400 mg of elemental zinc per day for a desired period of time.

Kits

The neuroprotective composition and methods described herein can be embodied as parts of a kit or kits. A non-limiting example of such a kit comprises the ingredients for preparing a neuroprotective composition, namely saline, a zinc chelator, and an acid, in separate (or at least two or more) containers, where the containers may or may not be present in a combined configuration. Many other kits are possible, such as kits comprising a suitable buffer system or pharmaceutically acceptable additive in a separate container. In certain embodiments, the kits further comprise a means for perfusing the composition to an anatomical location, such as sterile tubing. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive, CD-ROM, or diskette. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

### Example 1—Animal Model

The neuroprotective composition was tested in an animal model. The results of the test show that the application of a neuroprotective composition (acidic pH saline plus a zinc chelator) significantly reduces and prevents brain injury after TBI.

Animals were subjected to TBI that was modeled by weight-drop injury. After the TBI, the injured area of the brain was continuously perfused or irrigated with saline for 4 hours. In the control group, only physiological saline (pH˜7.0-7.4) was applied. In the hypertonic saline group, 20% glucose was included in physiological saline (pH˜7.0-7.4). In the treatment group, CaEDTA (5 mM) was added as a zinc chelator in the irrigation saline. The pH of the saline solution including the zinc chelator was then adjusted to about 4.3 by the addition of HCl.

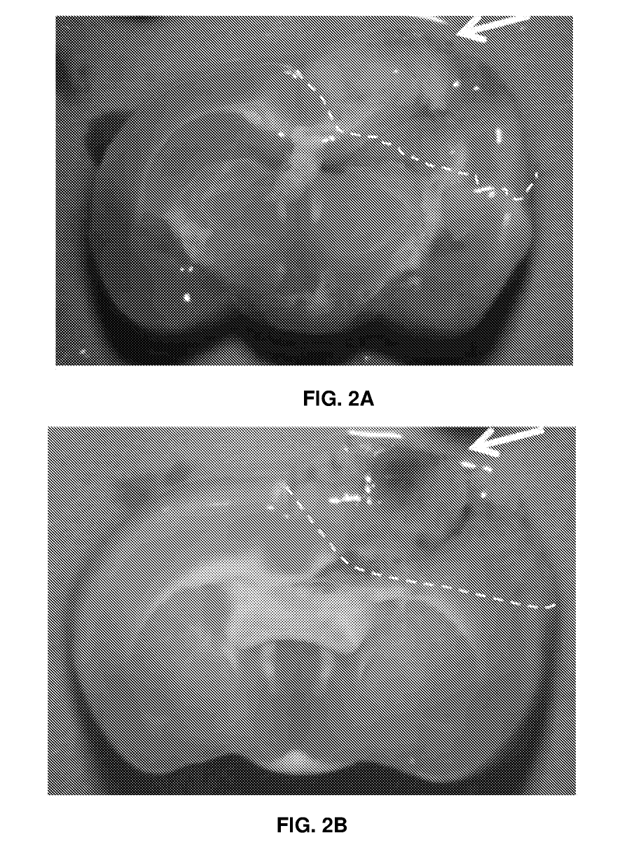

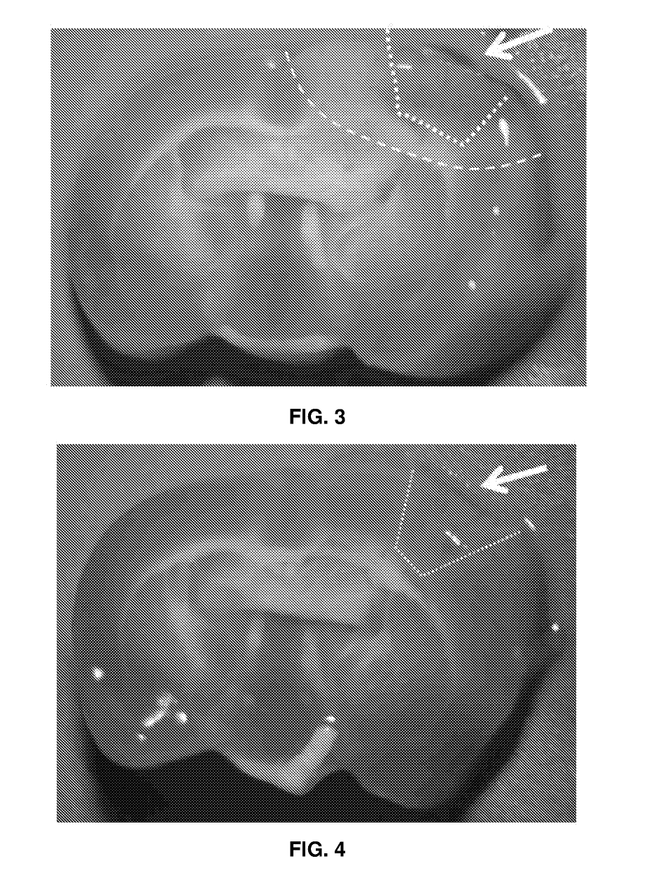

FIGS. 2A-2B show cross-sections of the brain following TBI and perfusion with physiological saline. The arrow indicates the direct impact area by the TBI. The brain area above the white dashed line is the damaged brain. As seen in FIGS. 2A-2B, damaged brain areas are markedly swollen. FIG. 3 shows a cross-section of a post-TBI brain that was continuously perfused with hypertonic saline containing 20% glucose immediately following the TBI. The addition of 20% glucose makes the saline become hypteronic. Brain area above the white dashed line is the damaged brain. The area included in the upper dashed line (at upright corner) is the area that is directly hit or impacted by the TBI. The brain damage between the two dashed lines in FIG. 3 is likely caused by the secondary impact of the TBI. Damaged brain areas are markedly less swollen compared to the brain section shown in FIGS. 2A-2B.

FIG. 4 shows a cross-section of the brain following TBI and continuous perfusion with hypertonic saline containing 20% glucose and CaEDTA at a low pH (pH˜4.3). The arrow indicates the direct impact area by the TBI. Immediately after the TBI, the surface of the brain was continuously perfused for 2 hours. The brain was then continuously perfused with hypertonic saline and CaEDTA for another 2 hours. The acidic saline was used to keep Zn²⁺ as free Zn²⁺. CaEDTA was the Zn²⁺ chelator that removed Zn²⁺ ions from the inside of neurons/cells. This result shows that brain damage by TBI was limited to the direct impact area.

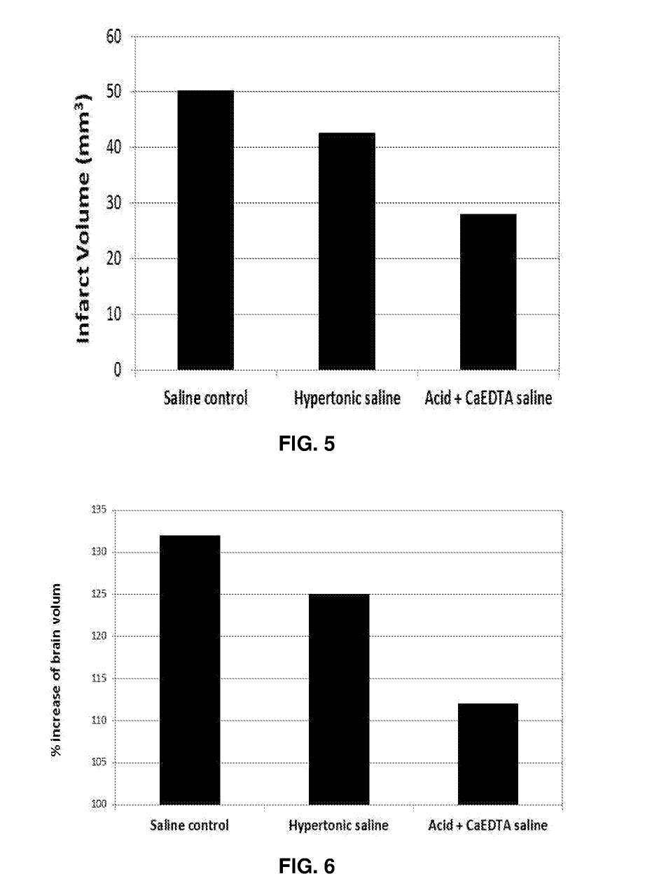

FIG. 5 shows that the neuroprotective composition reduces brain edema or swelling caused by TBI. The brain edema, expressed in % increases, was calculated by dividing the injured side by the normal side of the brain. As seen in FIG. 5, there was an average 132% increase of brain volume in the saline control, 125% increase of the brain volume in the hypertonic saline treatment, and about 112% increase of the brain volume in the acidic hypertonic saline treatment. The acidic hypertonic saline had a pH of 4.3 CaEDTA, and 20% glucose.

Furthermore, the neuroprotective composition reduced the infarct volume or brain damage area caused by traumatic brain injury, compared to a physiological salien control and a hypertonic saline at neutral pH, as shown in FIG. 6.

### Example 2—Zinc Precipitation

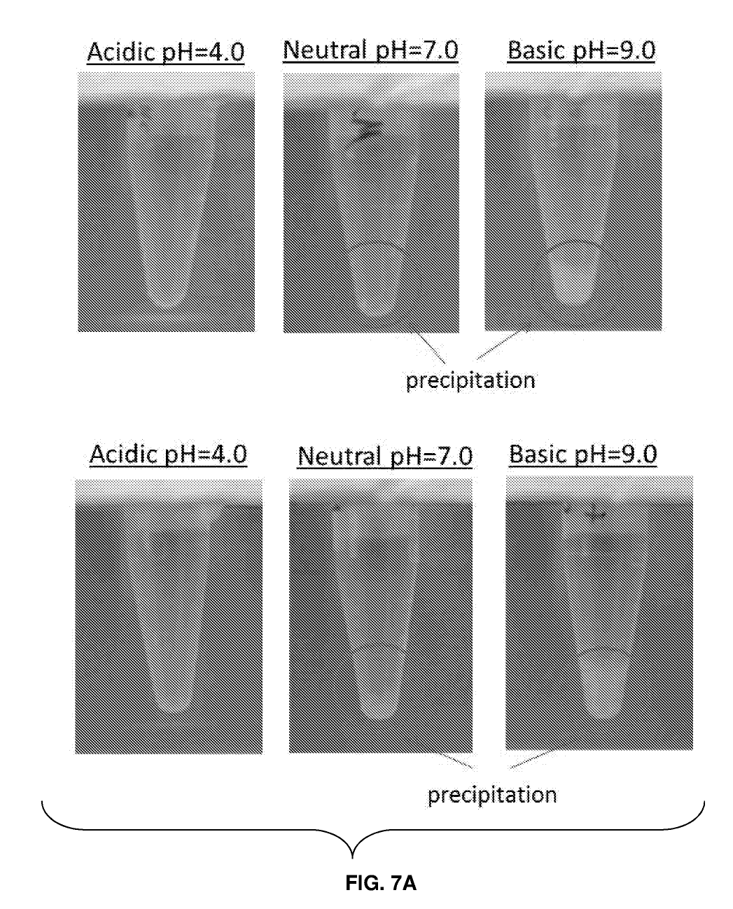

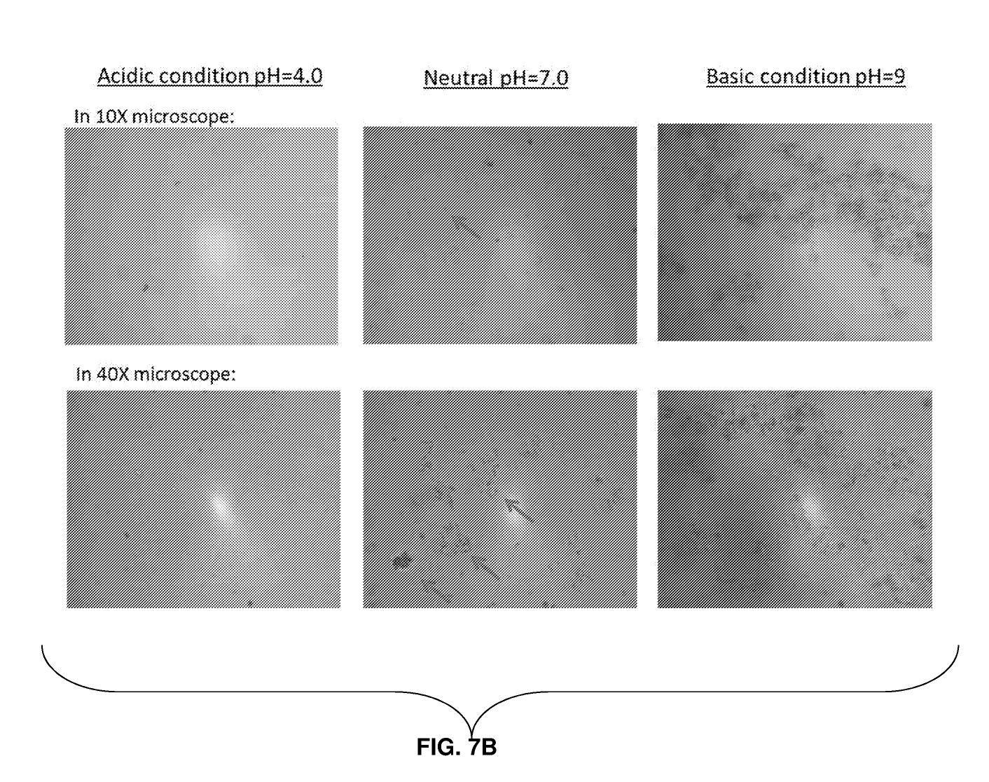

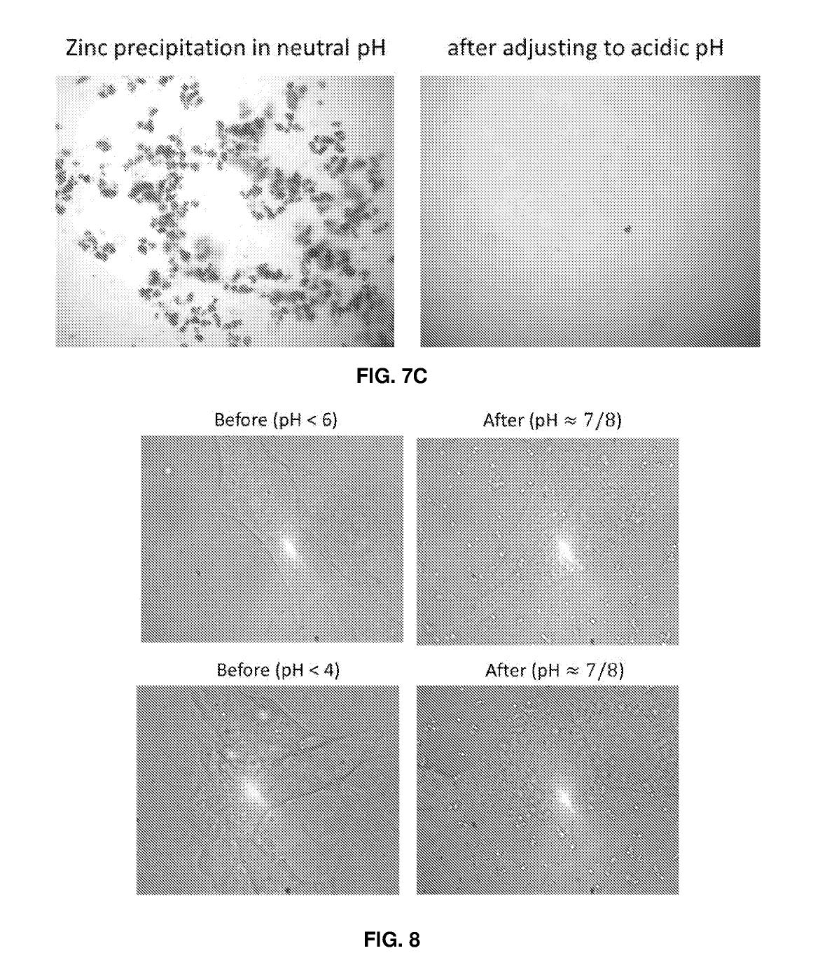

Zinc dissolution in acidic solution and zinc precipitation or crystallization in neutral or basic solution is shown in the photographs in FIGS. 7A-7C. The tests were carried out by the application of zinc into solutions. The results were observed with naked eyes (FIG. 7A) and with a microscope (FIG. 7B). FIG. 7C shows zinc precipitation in neutral pH, which was dissolved when pH was adjusted to an acidic pH. These images demonstrate that zinc is generally soluble under acidic conditions and tends to crystallize or precipitate under neutral and basic conditions.

In vitro precipitation of zinc was analyzed using different pH treatments in HeLa cells, the most commonly used human cell line. HeLa cells were used to test the cell damage caused by zinc precipitation or crystallization, at the single cellular level. Cells were first treated with acidic solution, which caused intracellular free zinc accumulation due to zinc dissociation from its binding proteins at low pH. Cells were then treated in neutral to slightly basic solution. Under neutral or slightly basic conditions, there were a large amount of debris, fragmentations, and broken cells visible. For those cells still having cellular morphology under this condition, as shown in the images (pH=7/8) in FIG. 8, there were apparent massive vacuoles forming inside cells, indicating dying cells.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A neuroprotective composition comprising saline and a zinc chelator, wherein the composition is at a pH ranging from about 2 to about 7.

2. The neuroprotective composition of claim 1, wherein the pH ranges from about 4 to about 5.

3. The neuroprotective composition of claim 1, wherein the zinc chelator comprises CaEDTA.

4. The neuroprotective composition of claim 1, wherein the neuroprotective composition is isotonic.

5. The neuroprotective composition of claim 1, wherein the neuroprotective composition is hypertonic.

6. The neuroprotective composition of claim 1, further comprising glucose.

7. The neuroprotective composition of claim 6, wherein the glucose is present in an amount sufficient to make the neuroprotective composition hypertonic.

8. The neuroprotective composition of claim 6, wherein the glucose is present at a concentration of about 20% by weight.

9. The neuroprotective composition of claim 1, wherein the zinc chelator is selected from the group consisting of: ethylenediaminetetra-acetic acid (EDTA); 1,3-diaminopropane-N,N,N′,N′-tetraacetic acid (DTPA); N,N,N′,N′-tetrakis(2-pyrdiylmethyl) ethylenediamine (TPEN); 1,10-phenanthroline; clioquinol; diethyldithiocarbamate (DEDTC), 2,3-dimercapto-1-propanesulfonic acid (DMPS); ethylenediamine-N,N′-diacetic-N,N′-di-B-propionic acid (EDPA); 1,2-dimethyl-3-hydroxy-4-pyridinone (DMHP); 1,2-diethyl-3-hydroxy-4-pyridinone (DEHP); ethylmaltol (EM), 4-(6-Methoxy-8-quinaldinyl-aminosulfonyl)benzoic acid potassium salt (TFLZn); dithiozone; N-(6-methoxy-8-quinolyl)-para-toluenesulfonamide (TSQ); carnosine; deferasirox; trans-1,2-cyclohexane-diamine-N,N,N′,N′-tetraacetic acid (CyDTA); dihydroxyethylglycine (DHEG); 1,3-diamino-2-hydroxypropane-N,N,N′,N′-tetraacetic (DTPA-OH); ethylenediamine-N,N′-diacetic acid (EDDA); ethylenediamine-N,N′-dipropionic acid (EDDP); ethylenediamine-N,N′-bis(methylphosphonic) acid (EDDPO); N-hydroxy-ethylenediamine-N,N′,N′-triacetic acid (EDTA-OH); ethylenediaminetetra(methylenephosphonic) acid (EDTPO); N,N′-bis(2-hydroxybenzyl)ethylenediamine-N,N′-diacetic acid (HBED); hexamethylene-1,6-diaminetetraacetic acid (HDTA); hydroxyethyliminodiacetic acid (HIDA); iminodiacetic acid (IDA); methyl-EDTA, nitrilotriacetic acid (NTA); nitrilotripropionic acid (NTP), nitrilotrimethylenphosphonic acid (NTPO); 7,19,30-trioxa-1,4,10,13,16,22,27,33-octaazabicyclo[11,11,11] pentatriacontane (O-Bistren); triethylenetetraaminehexaacetic acid (TTHA); ethyleneglycol bis(2-aminoethyl ether)-N,N,N′,N′-tetraacetic acid (EGTA); dimercaptosuccinic acid (DMSA); deferoxamine; dimercaprol; zinc citrate; combinations of bismuth and citrate; penicilamine; succimer; etidronate; ethylenediamine-di (O-hydroxyphenylacetic acid) (EDDHA); trans-1,2-cyclohexanediaminetetraacetic acid (CDTA); N-(2-hydroxyethyl) ethylenedinitrilotriacetic acid (HEDTA); N-(2-hydroxyethyl) iminodiacetic acid (HEIDA); 9-(O-carboxyphenyl)-2,7-dichloro-4,5,-bis[bis(2-pyridylmethyl)-aminomethyl]-6-hydroxy-3-xanthone; 9-(O-carboxyphenyl)-4,5-bis[bis(2-pyridylmethyl)-aminomethyl]-6-hydroxy-3-xanthanone; 9-(O-carboxyphenyl-2-chloro-5-[2-{bis(2-pyridylmethyl)aminomethyl}-N-methylaniline]-6-hydroxy-3-xanthanone; calprotectin; zinc fingers; lactoferrin; ovotransferrin; conalbumin; salts thereof; and combinations thereof.

10. The neuroprotective composition of claim 1, comprising a buffer that maintains the pH of the composition in the range of from about 2 to about 7.

11. The neuroprotective composition of claim 1, comprising a buffer that maintains the pH of the composition in the range of from about 4 to about 5.

12. The neuroprotective composition of claim 1, further comprising an additive selected from the group consisting of: contrast agents, secondary chelators, antibiotics, antiseptics, antifungals, growth factors, nucleic acids, proteins, chemotherapeutic agents, vitamins, bone resorption inhibitors, stem cells, dyes, opacifying agents, drug delivery vehicles, diagnostic agents, materials designed to release ions, and other small molecule or biological drugs.

13. A method for treating an open wound or injury, the method comprising:
dissolving a zinc chelator in a saline solution;
adjusting the pH of the saline solution to a pH of from about 2 to about 7, to produce an acidic composition; and.
administering the acidic composition to an open wound or injury to treat the open wound or injury.

14. The method of claim 13, wherein the open wound or injury is a traumatic brain injury.

15. The method of claim 13, wherein swelling of the open wound or injury is reduced.

16. A method of treating a traumatic brain injury comprising:
lowering the pH of injured brain tissue;
chelating free zinc ions present in the injured brain tissue with one or more zinc chelators; and
allowing the pH of the injured brain tissue to return to neutral or near-neutral.

17. The method of claim 16, further comprising treating the injured brain tissue with a hypertonic solution to reduce swelling of the injured brain tissue.

18. A method for treating an open wound or injury, the method comprising:
covering a wounded area of tissue with material soaked in a neuroprotective composition of claim 1 for a first period of time; and
optionally, washing the wounded area with physiological saline for a second period of time.

19. The method of claim 18, wherein the wounded area of tissue comprises brain tissue.

20. The method of claim 18, wherein swelling in or around the open wound or injury is reduced.

21. A method for treating an injury comprising:
washing or irrigating an injury with a composition of claim 1 for a first period of time; and,
washing or irrigating the injury with normal saline for a second period of time, to treat the injury.

22. The method of claim 21, wherein the injury is a traumatic brain injury (TBI).

23. A method of preventing an infection comprising:
washing an injury or open wound of a subject with a neuroprotective composition of claim 1 for a sufficient period of time to treat the injury or open wound; and
treating the subject with an antibiotic to prevent an infection in the injury or open wound.

24. A kit for treating a wound or injury comprising:
a first container housing a saline solution; and
a second container housing a zinc chelator.

25. The kit of claim 24, further comprising a third container housing an acid or buffer system.
